(19) United States
(12) Patent Application Publication
(10) Pub. No.: US 2006/0216342 A1
(43) Pub. Date: Sep. 28, 2006

(54) Micelle delivery system loaded with a pharmaceutical agent

(76) Inventors: Vladimir P. Torchilin, Charlestown, MA (US); Anatoly N. Lukyanov, Brighton, MA (US); Zhonggao Gao, Bookline, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/553,612

(22) PCT Filed: Apr. 18, 2003
(86) PCT No.: PCT/US03/12042

§ 371 Date: Oct. 18, 2005

(51) Int. Cl. A61K 39/395; A61K 31/555; A61K 31/4745; A61K 31/4709; A61K 31/137; A61K 31/195; A61K 31/198; A61K 31/426; A61K 31/407

(52) U.S. Cl. 424/450; 424/178.1; 514/185; 514/283; 514/369; 514/410; 514/566; 514/682; 514/651; 514/314

(57) ABSTRACT

The invention is directed to an improved drug delivery system that includes a micelle, comprising polyethylene glycol and a lipid component, and a pharmaceutical agent dispersed in the lipid component. The delivery system may also include a targeting ligand. The micelle delivery system of the invention is capable of stabilizing, inter alia, poorly soluble pharmaceutical agents and increasing their delivery efficacy. Appropriate pharmaceutical agents useful in the system of the invention include anti-inflammatory agents, agents for photodynamic therapy, anti-tumor agents, anti-neoplastic agents, anti-metastatic agents, and imaging agents, as well as hydrophobized derivatives thereof. Specifically, the pharmaceutical agent can be porphyrin, chlorine-6-trimethyl ester, tamoxifen, paclitaxel, 1,3-bis(2-chloroethyl)-1-nitrosourea, camptothecin, ellipticine, rhodamine, dequalinium, diphenylhexatriene, vitamin K3, diethylene triamine pentaacetic acid, or a functional derivative thereof. The micelles in the system of the invention have low critical micellar concentration and high kinetic stability, which provides great advantages in biodistribution, for example, accumulation at the site of a tumor.

### STATEMENT REGARDING FEDERALLY SPONSORED RESEARCH OR DEVELOPMENT

Part of the work leading to this invention was carried out with United States Government support provided under a grant from the National Institutes of Health, Grant No. GM602000-03. Therefore, the U.S. Government has certain rights in this invention.

### CROSS REFERENCE TO RELATED APPLICATIONS

N/A

### BACKGROUND OF THE INVENTION

The requirements placed on pharmaceutical drug carriers for intravenous administration include small size, biodegradability, good loading capacity, high content of the drug in a final preparation, prolonged circulation, and ability to accumulate in required areas. These requirements are reasonably well met by some drug carriers (microcapsules, liposomes) used predominantly for water-soluble drugs (Muller, 1991; Lasic et al., 1995; and Cohen et al., 1996). Although liposomes can entrap poorly soluble drugs in the hydrophobic bilayer, their loading capacity is limited because of possible membrane destabilization. Thus, the development of drug carriers displaying all the named properties specifically for the delivery of poorly soluble pharmaceutical agents continues to represent a challenge.

Low solubility in water tends to be an intrinsic property of many drugs, including anti-cancer agents, which often represent polycyclic compounds (Shabner et al., 1990). The membrane permeability and efficacy of such drugs increases with increasing hydrophobicity (Yokogawa et al., 1990; and Hageluken et al., 1994). On the other hand, parenteral administration of those intrinsically hydrophobic agents is associated with some problems. Thus, intravenous administration of aggregates formed by undissolved drug in aqueous media can cause embolization of blood capillaries (≦5 μm) before the drug penetrates a tumor (Fernandez et al., 2001). Additionally, the low solubility of hydrophobic drugs in combination with excretion and metabolic degradation hinders the maintenance of therapeutically significant systemic concentrations.

For example, derivatives of porphyrin are used as agents for photodynamic therapy (PDT). This approach employs a combination of light and chemicals and is used in the treatment of cutaneous T-cell lymphoma and cavitary tumors. The use of PDT is complicated by some undesired side effect caused by accumulation of PDT agents in non-target organs (Dalla Via et al., 2001). Poor solubility of some porphyrin derivatives is also an issue (Songca et al., 2000) and requires increased quantities of the drug to be used to achieve a therapeutic effect, which in turn, increases said effects.

Tamoxifen is a drug for breast cancer chemotherapy with poor solubility in water (Ferlini et al., 1997). This drug has been used with varying degrees of success to treat a variety of estrogen receptor positive carcinomas such as breast cancer, endometrial carcinoma, prostate carcinoma, ovarian carcinoma, renal carcinoma, melanoma, colorectal tumors, desmoid tumors, pancreatic carcinoma, and pituitary tumors (Furr et al., 1984). Long term tamoxifen therapy causes some side effects such as endometrial cancer and drug resistance (Johnston et al., 1997).

Taxol (or paclitaxel) is an anticancer drug that causes stabilization of microtubules and thus interferes with cellular progress through mitosis and arresting cell replication. An important hinderance to its effective use, however, is its poor solubility in water and in most pharmaceutically acceptable solvents. It is normally administered intravenously by dilution into saline of the drug dissolved or suspended in polyoxyethylated caster oil. This carrier has been reported to induce an anaphylactic reaction in a number of patients (Sarosy et al., 1993). In addition, paclitaxel is a toxic drug and therefore, large doses may cause severe toxic reactions (Arbuck et al., 1993).

BCNU {1,3-bis(2-chloroethyl)-1-nitrosourea} is well known for its anti-tumor properties and, since 1972, it has been charted by the National Cancer Institute for use against brain tumors, colon cancer, Hodgkins disease, lung cancer and multiple myeloma. However, the efficient use of this anticancer drug is also compromised by its low solubility (Layton et al., 1984).

The use of all of the above mentioned pharmaceutical agents suffers from their poor solubility in water, accumulation in non-target organs and associated toxicity. The development of improved delivery vehicles for such compounds is clearly desirable and would make it possible to overcome the above mentioned problems and substantially enhance the efficiency of, for example, cancer treatment.

### BRIEF SUMMARY OF THE INVENTION

The present invention is directed to an improved drug delivery system comprising a targeted form of a polyethyleneglycol (PEG)/lipid-conjugated micelle, which is capable of stabilizing poorly soluble pharmaceutical agents and of increasing their delivery efficacy. The micelles of the invention can also be conjugated with modified disease-specific ligands for intracellular delivery.

The invention is specifically targeted for the delivery of pharmaceutical agents into the required areas of the body; the pharmaceutical agent delivery system according to the invention has high loading capacity, controlled release and good compatibility between the core forming micelle and the incorporated pharmaceutical agent. The characteristic features of the targeted micelle of the invention are, inter alia, its high stability both in vitro and in vivo, which constitutes having an extremely low critical micellar concentration (CMC) and a high kinetic stability. The improved delivery system of the invention can be used for solubilizing some of the most important poorly soluble pharmaceutical agents and for improving systemic administration of these agents. This invention is a colloidal dispersion of micelles with a diameter in the range between 5 nm to 100 nm loaded with pharmaceutical agents that have, e.g., anti-inflammatory, anti-tumor, anti-metastatic, anti-neoplastic, imaging, or photodynamic activity. The purpose of the invention is to provide, for example, better bioavailability of pharmaceutical agents, protect them against destructive environment upon in vivo administration and promote their accumulation in, e.g., a tumor cell.

### BRIEF DESCRIPTION OF THE FIGURES

Other features and advantages of the invention will be apparent from the following description of the preferred embodiments thereof and from the claims, taken in conjunction with the accompanying drawings, in which:

FIG. 1A is a schematic structure of PEG-PE micelles containing a small addition of the pNP-PEG-PE component; FIG. 1B is a coupling of amino group-containing ligands (antibodies) with pNP groups;

FIG. 2 shows the incorporation of porphyrin into PEG-PE micelles;

FIG. 3 shows the encapsulation of tamoxifen into PEG-PE micelles: (1) Tamoxifen concentration before filtration; (2) Concentration of Tamoxifen in PEG-PE micelles after filtration; (3) Concentration of Tamoxifen without PEG-PE after filtration;

FIG. 4A is a size distribution of PEG-PE micelles with tamoxifen; and FIG. 4B is a size distribution without tamoxifen;

FIG. 5A shows the attachment of 2C5 antibodies to PEG-PE micelles via micelle-incorporated pNP-PEG-PE; and FIG. 5B shows 2C5 attachment yield as a function of pNP-PEG-PE content in the micelle;

FIG. 6 shows (Top) size distribution of “plain” PEG-PE/pNP-PEG-PE micelles (left panel), and same micelles after the attachment of 2C5 (middle panel) or 2G4 (right panel) and (Bottom) freeze-fracture electron images of plain PEG-PE/pNP-PEG-PE micelles (A), and 2C5-immunomicelles of the same composition (B);

FIG. 7A shows binding of 2C5-immunomicelles to a monolayer of nucleosomes and FIG. 7B shows binding of 2G4-immunomicelles to a monolayer of myosin;

FIG. 8 shows microscopy data on the binding of Rh-labeled 2C5-immunomicelles to EL 4 T lymphoma cells (top panel), LLC cells (middle panel) and BT20 mammary adenocarcinoma cells (bottom panel). Left images—bright field light microscopy; Right images—fluorescent microscopy;

FIG. 9 shows blood clearance of plain micelles and 2C5-immunomicelles in mice;

FIG. 10A shows the accumulation of PEG-PE micelles in subcutaneous LLC tumor in mice at different time points and FIG. 10B shows the accumulation of free and micellar taxol in LLC tumor at the same time points;

FIG. 11 shows inhibition of LLC tumor growth in mice with different taxol preparations;

FIG. 12 shows the results of blood clearance experiments;

FIGS. 13A and 13B show the accumulation of micelles prepared from PEG₇₅₀-PE and PEG₂₀₀₀-PE in LLC; FIG. 13A depicts the pharmacokinetics and FIG. 13B depicts AUC; and

FIGS. 14A and 14B show the accumulation of micelles in EL4 cells; FIG. 14A depicts the pharmacokinetics and FIG. 14B depicts AUC.

### DETAILED DESCRIPTION OF THE INVENTION

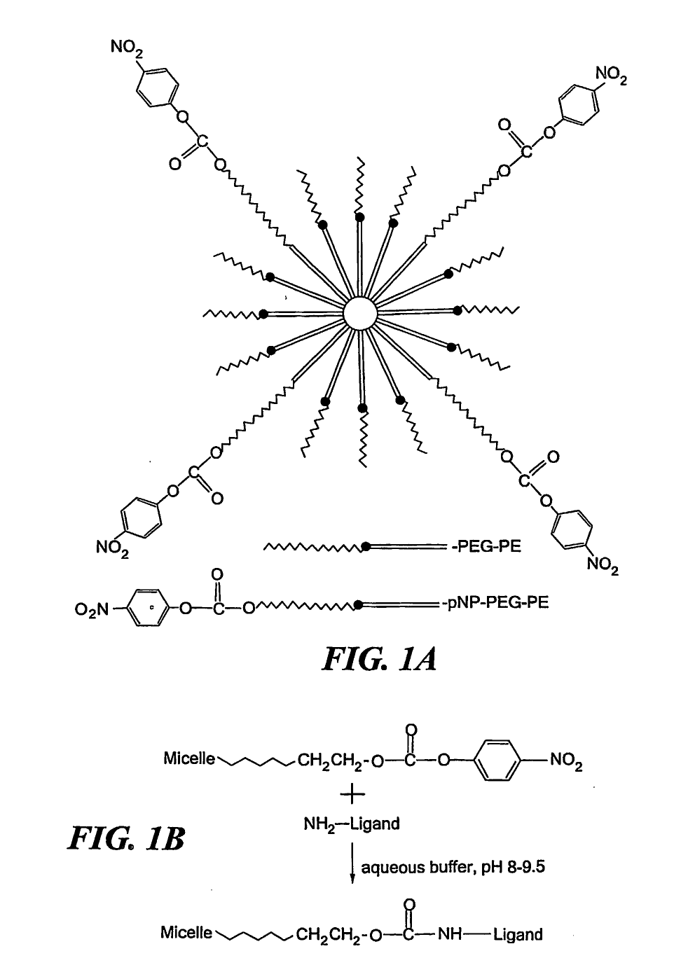

Micelles are spherical colloidal nanoparticles, into which many amphiphilic molecules self-assemble. In water, hydrophobic fragments of amphiphilic molecules form the core of a micelle, which may then be used as a cargo space for poorly soluble pharmaceutical agents (Lasic, 1992; and Muranishi, 1990). An exemplary micelle structure and ligand attachment are illustrated in FIG. 1. Micelle encapsulation can increase the bioavailability of poorly soluble drugs, protect them from destruction in biological surroundings, and beneficially modify their pharmacokinetics and biodistribution (Hammad et al., 1998). Because of their small size (in the range between, 5 to 100 nm), micelles demonstrate spontaneous accumulation in pathological areas with leaky vasculature, such as infarct zones (Palmer et al.) and tumors (Gabizon, 1995; Yuan et al., 1994). This phenomenon is known as the enhanced permeability and retention (EPR) effect (Maeda et al., 2000; 2001).

Since microparticulate drug carriers are removed from the blood via the opsonization-mediated phagocytosis by cells and organs of the reticuloendothelial system (Senior, 1987), the micelle corona formed by hydrophilic polymer blocks provides longevity to micelles in vivo by preventing their opsonization and capture (Torchilin et al., 1995). Amphiphilic polymers have a low critical micelle concentration (CMC), which makes polymeric micelles stable and prevents their rapid dissociation in vivo. The use of lipid moieties as hydrophobic blocks provides an additional stability, since the existence of two hydrocarbon chains contributes considerably to the increased hydrophobic interactions in the micelle's core. Micelles prepared from conjugates of polyethyleneglycol (PEG) and diacyllipids, such as phosphatidylethanolamine (PE) are of particular interest (Trubetskoy et al., 1995).

However, although micelles made of polymer-lipid conjugates have been prepared and used in some circumstances to solubilize specific substance, it has not been possible to predict what type of micelle could be used for a particular poorly soluble compound to form a stable composition. In other words, the stability of a specific micelle forming mixture and a specific poorly soluble compound cannot be predicted a priori.

Micelles made from PEG-PE conjugates were first disclosed in the study performed by Trubetskoy et al. (Acad Radiol, 1996). This study concluded that PEG-PE micelles can incorporate certain insoluble and amphiphilic agents and prolong their circulation in vivo by avoiding the reticuloendothelial system (RES). However, some micelle/insoluble agent combination caused micelle aggregation or phase separation. Thus, this experiment shows that it is not possible to predict the stable incorporation of any given poorly soluble compound into micelles.

U.S. Pat. No. 6,322,810 (Alkan-Onyuksel et al., 2001) discloses the use of micelles prepared from distearoyl-phosphatidylethanolamine covalently bonded to PEG (PEG-DSPE) for the improved delivery and presentation of amphipathic peptides for therapeutic, diagnostic and cosmetic use. U.S. Pat. No. 6,338,859 (Leroux et al., 2000) and references therein disclose the use of polymer micelles for the delivery of poorly soluble drugs incorporated into their hydrophobic core.

These disclosures demonstrate that incorporation of poorly soluble drugs into lipid-polymer micelles is possible and can provide better biodistribution characteristics and enhance tumor accumulation of these specific micelle-incorporated drugs. However, the compatibility between a loaded drug and a specific micelle-forming component depends on such drug characteristics as polarity, hydrophobicity and charge and, thus, is unpredictable. The possible outputs of micelle forming compound/drug interaction include formation of separated micellar and crystalline drug phases, formation of liposomes and/or even larger polymer/drug aggregates, or disintegration of micellar phase under the influence of poorly soluble drugs. Moreover, the loading efficacy of some poorly soluble drug may be below practical limit. Thus, there clearly remains a need for a new and improved drug delivery system for some of the most important currently used, for example, anti-cancer drugs.

The micelles of the invention specifically target disease-affected organ and/or tissue. Numerous examples described above show that the clinical potential of many anti-cancer drugs may not be fully realized because of poor solubility of these drugs in water. Micelles of the invention overcome this problem and substantially enhance, for example, anti-tumor efficacy of many existing drugs.

Pharmaceutical agents used in accordance with the invention can be anti-inflammatory agents, anti-tumor agents, anti-metastatic agents, anti-neoplastic agents, imaging agents, or agents for photodynamic therapy. In particular, exemplary pharmaceutical agents include, but are not limited to, chlorin e6 trimethyl ester (modified porphyrin), tamoxifen, paclitaxel (taxol) and BCNU {1,3-bis(2-chloroethyl)-1-nitrosourea}, camptothecin, ellipticine, rhodamine, dequalinium, diphenylhexatriene, vitamin K3 and functional derivatives or hydrophobized derivatives (Lambert, 2000; and Torchilin, 2000, Curr Pharm Biotenchnol) thereof. Additionally, other types of agents used for imaging or diagnostics include, but are not limited to, chelating agent diethylene triamine pentaacetic acid (DTPA). DTPA is an agent used to firmly chelate radioactive metals such as 111-In or 99m-Tc for gamma-imaging. Heavy metals with paramagnetic properties may also be used, for example, galladium (Gd) or manganese (Mn) used for magnetic resonance imaging. These metals are modified (using known protocols) with hydrophobic “tails” such as steryl (e.g., sterylamine (SA)) or diacyllipid (e.g., phosphatidyl ethanolamine (PE)) to give DTPA-SA and DTPA-PE. These hydrophobized chelators have been incorporated into micelles and loaded with metals for gamma- or MR-imaging. (Torchilin, 2000, Curr Pharm Biotechnol; and Trubetskoy et al., 1996, Acad Radiol).

It is also known that a large number of examples that target particulate delivery systems substantially enhances their efficiency (Torchilin, 2000, Eur J Pharm Sci). The targeting micelles of the invention increase the bioavailability of poorly soluble pharmaceutical agents, provide protection of the drug against destructive environment upon in vivo administration and/or provide for a safe and efficient intracellular delivery of pharmaceutical agents. Delivery of drugs directly to the site of their action is vastly preferable to systemic administration.

Additionally, targeted delivery by micelles may reduce both transient toxic levels of a drug at the beginning of administration, and the number of doses while not exceeding a toxic threshold (Langer, 1998). Targeted drug delivery also promotes patient compliance by decreasing the number of doses required for therapy completion. Patient non-compliance is responsible for approximately 10% of hospital admission (Kefalides, 1998). Increasing patient compliance and decreasing side effects could substantially reduce hospital admissions, prevent deaths, and improve patient quality of life.

Several other properties of the micelles of the present invention add to their novelty and improvement over the art. The micelles of the invention have the ability to attach a variety of targeted ligands. The methods for ligand attachment disclosed allow preparing micelles targeted against a broad variety of tissues and organs with abnormalities associated with different diseases.

The micelles are biocompatible and biodegradable. It has been shown that the majority of proposed micelle-forming compounds have low toxicity and are completely biodegradable. In particular, low toxicity of oligo(poly)ethyleneglycol-based surfactants has been extensively reported. This means that the micellar drug delivery system prepared from PEG-lipid conjugates will be safe to use.

The micelles are self-assembling. Unlike many alternative particulate delivery systems, which require complicated technological processes to prepare, the micelles form spontaneously under appropriated conditions. This means that it would be very easy to develop technology for mass production of the delivery system disclosed.

The lipid-polymer micelles of the invention also have low critical micelle concentration (CMC) values and high stability in blood. The CMC is the concentration of a monomeric amphiphile at which micelles appear. The micelles of the invention reach a rapid dynamic equilibrium where the size of the micelles does not change. While a number of pharmaceutical micelle-forming compounds with low toxicity and high solubilization power are available, these conventional surfactants, however, have CMC values in the millimolar range (Rosen, 1989) and may dissociate upon being diluted to therapeutically acceptable concentrations. Upon in vivo administration, this may result in micelle collapse in a large blood volume with a subsequent precipitation of the encapsulated drug, i.e., sharp decrease in its bioavailability and ability to penetrate biological barriers. The loaded PEG-lipid conjugate micelles of the invention, unlike micelles formed from conventional detergents, are stable upon dilution to therapeutically applicable concentration.

The micelles of the invention have the ability to be loaded with large quantities of poorly soluble pharmaceuticals. Many formulations disclosed may be loaded with poorly soluble pharmaceutical agents with higher efficiency compared to alternative systems, such as oil-in-water emulsions or liposomes.

The micelles also have long circulation times. The water-soluble polymer corona protects the micelles from uptake by a non-target organ, thus allowing the micelles to circulate in the blood stream for a long time and accumulate in the target organs with higher efficiency.

In another aspect of the invention, the micelles used with nucleosome-specific antibodies have the ability to target many tumors. Targeted micelles loaded with poorly soluble anticancer drugs, e.g., taxol, and modified with broadly specific anticancer antibodies can provide an efficient mean for drug delivery into a variety of tumors.

The micelles of the invention have the ability of intracellular drug delivery if transduction proteins/peptides are attached to the micelles. This feature is important because many therapeutic agents bind to receptors and act only if they are delivered inside a cell.

In yet another aspect, the micelles of the invention have the ability to deliver a drug into a tumor via an enhanced permeability and retention effect (EPR). During the last several years, it was clearly demonstrated that the passive accumulation of drug carriers in areas with leaky vasculature, such as tumors, depends on the vasculature pore cut-off size (Gabizon, 1995; Maeda et al., 2000). In particular, it has been shown that different tumors possess different vascular permeability and, in certain cases, this permeability can be rather low (small cut-off size of 200 nm or less), which prevents many drug carriers including long-circulating liposomes from accumulation in such tumors (Weissig et al., 1998). The size of the micelles disclosed perfectly suits them for delivery of drugs to a tumor, utilizing an enhanced permeability retention effect.

There are many considerations for determining the stability of different blocks of micelles: (a) an increase in the length of the hydrophobic block for a given length of the hydrophilic block causes a noticeable decrease in CMC value and increase in micelle stability; (b) an increase in the length of the hydrophilic block for a given length of the hydrophobic block results in only a small rise in the CMC value; (c) an increase in the molecular weight of the unimer for a given hydrophilic/hydrophobic ratio of mass causes some decrease in the CMC value; (d) in general, the CMC value for tri-block copolymers is higher than for di-block copolymers at the same molecular weight and hydrophilic/hydrophobic ratio. (Torchilin, 2001, J. Control. Release) This determines the efficacy of drug loading into the micelle, the drug release profile and the micelle stability.

Other considerations require attention in loading a micelle, with an appropriate poorly soluble pharmaceutical agent for its efficacy. The compatibility between the micelle and the agent is based on the agent's characteristics such as polarity, hydrophobicity and charge. To assess compatibility between the polymer and solubilized drug, the Flory-Huggins interaction parameter may be used (Allen et al., 1999). This parameter consists of the Scatchard-Hildebrand solubility parameter of the core-forming polymer block and the molar volume of the solubilized drug (Torchilin, 2001, J. Control. Release). The lower the parameter, the greater the compatibility between the drug and the micelle core.

However, excessive stabilization of drug-bearing polymeric micelles may negatively influence drug efficacy and bioavailability, since the drug would not release from such micelles. Thus, the real optimization of micelle properties as drug carriers involves a proper balance between micelle stability and their ability to dissociate or degrade.

Preparation of Micelles

The micelles can be prepared by any convenient method (see for example, (Torchilin, 2001, J Control Release) from amphiphilic components (such as lipidated polymer) combined with various poorly soluble pharmaceutical agent in a form of mechanical mixture (e.g., warming, shaking, stirring or ultrasound treatment) that spontaneously self-assembles in aqueous media. Alternatively, any known method of mixing solid ingredients may be applied. These methods include, for example, direct dissolution or dialysis of an amphiphile solution in a water-miscible organic solvent against aqueous medium (Torchilin, 2001, J Control Release). The organic solvent may be removed by evaporation. An excess of a poorly soluble agent that does not incorporate into micelles, may be removed by filtration and/or centrifugation. Resultant particles consist of a hydrophobic core made of water-insoluble fragments of amphiphilic molecules and poorly soluble drug surrounded by a protective shell formed by the water-soluble parts of amphiphilic molecules.

Conjugates of lipid residues with water-soluble polymers are another example of the micelle of the invention. In this case, the lipid and polymer parts are covalently attached to each other forming lipid-polymer block co-polymer. Examples of suitable lipids include, but are not limited to, saturated or non-saturated 18-28 carbon atoms long hydrocarbon chains fatty acids and phospholipids with saturated and non-saturated acyl chains with the length from 12 to 22 carbon atoms, linear or branched. Specifically, a lipid in accordance with the invention is a diacyllipid, e.g., phosphatidylethanolamine. Examples of water-soluble polymers include, but are not limited to, PEG with molecular weights in the range between 500 to 10,000 daltons with straight or branched polymer chains, preferably in the range between 1,000 to 8,000 daltons. In addition to amphiphilic components, lipids not carrying polymer part may also be included into particle composition yielding mixed micelles.

Attachment of Targeting Moieties to Micelle Surface

The surface of micelles according to the invention can be modified (for example, covalently) with any specific ligand, such as a protein, including antibody, or a peptide possessing the ability to specifically recognize certain cellular or molecular structures within the body of the patient. Specific ligands (for example, proteins or peptides) are attached to the micellar surface using any amphiphilic compound able to simultaneously perform two functions—incorporate and anchor into the micelle core via its hydrophobic part, and bind a specific ligand via a certain functional group (chemically reactive) located in an accessible fashion on a water-exposed hydrophilic portion of the compound. Such a compound can be added to a micelle-forming mixture in the process of micelle formation. An example of such a compound is para-nitrophenylcarbonyl-PEG-1, 2-dioleoyl-sn-glycero-3-phosphoethanolamine (pNP-PEG-PE) (Torchilin et al., 2001, Biochim Biophys Acta; Torchilin et al., 2001, Proc Natl Acad Sci USA), which was reported by us for liposome targeting. The chemical reactive groups on these compounds may be any group that reacts with proteins or peptides with formation of covalent bonds. The examples of such groups include, but are not limited to amino, thiol, carboxyl, epoxide, aldehyde, hydroxysuccinimide, sulfohydroxysuccinimide, isocyanate, maleimide and oxycarbonylimidazole. A specific ligand is attached to the micelles of the invention by co-incubation of specifically activated micelles with the ligand, allowing covalent bonds between the reactive micelle component and ligand to form.

Examples of specific ligands may include peptides, proteins, enzymes, lectins, -biotin, avidin, mono-, oligo-, and polysaccharides, hormones, cytokines, polyclonal and monoclonal antibodies including chimeric and humanized ones and their fragments. An example of targeting antibody may be an anticancer nucleosome-specific monoclonal antibody (Iakoubov et al., 1997). Several other monoclonal antibodies, which are known to react with tumor-associated antigens, may also be used. For example, the antibody may be an antibody that targets an antigen of tumor vascular endothelium. This type of targeting may be applicable to many types of solid tumors since many tumors require a blood supply, and endothelial cells are readily accessible from the bloodstream (Burrows et al., 1992). Antibody against cardiac myosin can be used to target micelles to infarcted areas of the heart muscle (Khaw et al., 1984). The micelles can be also (especially or additionally) modified with cell membrane translocation proteins and peptides. These proteins include HIV-1 TAT protein, Antennapedia protein (ANTP), and VP22 herpes virus protein (Fawell et al., 1994; Vives et al., 1997; Derossi et al., 1994; and Phelan et al., 1998). Translocating peptides include the “protein transduction domain” (PTDs) of all known translocating proteins as well as various synthetic translocating peptides (Plank et al., 1998; Mi et al., 2000). The use of these proteins and protein domains allows micelle and micelle-incorporated pharmaceuticals delivery across cellular membranes directly into the cell cytoplasm (Fawell et al., 1994; Plank et al., 1998; Mi et al., 2000; and Wagner, 1999).

### EXAMPLES

The following examples are presented to illustrate the advantages of the present invention and to assist one of ordinary skill in making and using the same. These examples are not intended in any way otherwise to limit the scope of the disclosure.

Exemplary Materials and Methods

Materials. Phosphatidylethanolamine (PE), poly(ethylene glycol)-2000-PE (PEG-PE) and PE-(lissamine-rhodamineB) (Rh-PE) were from Avanti Polar Lipids (Alabaster, Ala). p-Nitrophenylcarbonyl-PEG-PE was synthesized as described (Torchilin et al., 2001). Diethylenetriaminepentaacetic acid-PE conjugate (DTPA-PE) for radiolabeling micelles with ¹¹¹In was synthesized as in (Grant et al., 1989). RPMI medium 1640 (RPMI), Eagle's minimal essential medium (EMEM), modified Eagle's medium (DMEM), serum-free medium, and heat inactivated fetal bovine serum (FBS) were from Cellgro (Herndon, Va.). ¹¹¹In with specific radioactivity of 395 Ci/mg was from Perkin-Elmer Life Sciences (Boston, Mass.). Cancer-specific antinucleosome 2C5 is routinely produced in our laboratory. Taxol was purchased from Sigma (St. Louis, Mo.), and dissolved in Cremophor EL (BASF, Mount Olive, N.J.) mixed with ethanol (1:1 by volume) and then further in saline as described in (Sarosy et al., 1993). Antimyosin 2G4 was provided by Dr. B.-A. Khaw (Northeastern University, Boston, Mass.).

Cell cultures. Murine Lewis lung carcinoma (LLC) and EL4 T cell lymphoma (EL4), and human BT20 breast adenocarcinoma (BT20) cell lines were purchased from the American Type Culture Collection (Manassas, Va.). LLC and BT20 cells were maintained in DMEM with 10% of FBS, penicillin/streptomycin, pyruvate, L-glutamine and non-essential amino acids. EL4 cells were grown in RPMI with the same additives as above. Cells were grown at 37° C. in 5% CO₂.

Labeling of antibodies with carboxyfluorescein. A 50 μl aliquot of 2 mM 5-carboxyfluorescein (CF) succinimidyl ester (Molecular Probes, Eugene, Oreg.) in DMSO was added to 2 ml of a 12 pM solution of 2C5 in 50 mM Tris-buffered saline (TBS), pH 9.0. The mixture was incubated overnight at 4° C. Free CF was removed by dialysis against TBS.

Preparation of (immuno)micelles. A lipid film was prepared by removing chloroform from the mixed solution of PEG₂₀₀₀-PE and 2-to-8 mol % of pNP-PEG-PE under vacuum. To load micelles, taxol dissolved in methanol was added to a chloroform solution of PEG-PE and pNP-PEG-PE (1.5 mg of taxol per 80 mg of PEG-PE). When required, trace amounts of DTPA-PE and/or 0.5 mol % of Rh-PE were added to these preparations. To form micelles, the film was re-hydrated at 50° C. in a 5 mM Na citrate-buffered saline, pH 5.0, and vortexed for 5 min.

When required, 0.5 ml of a 12 μM solution of 2C5 or 2G4 in borate, pH 9.0 was added to 0.5 ml of pNP-PEG-PE-containing micelles with a PEG-PE concentration of 1.5 mM. The mixture was incubated for 3 h at room temperature (RT) and dialyzed against HBS, using cellulose ester membranes with a cut-off size of 300,000 Da (Spectrum Medical Industries, Rancho Dominguez, Calif.). To assess the quantity of the micelle-bound protein, CF-labeled antibody was used. Protein concentration was measured by associated fluorescence at an excitation wavelength of 490 nm and an emission of 520 nm on a F-2000 spectrofluorimeter (Hitachi, Japan). The micelle size was measured by dynamic light scattering using a N4 Plus Submicron Particle System (Coulter Corporation, Miami, Fla.) at PEG-PE concentration of 2-10 mM.

Freeze-fracture electron microscopy. The sample was quenched using the sandwich technique and liquid nitrogen-cooled propane. A cooling rate of 10,000K/sec avoids ice crystal formation and cryofixation-caused artifacts. The fracturing process was carried out in JEOL JED-9000 freeze-etching equipment (Jeol, Peabody, Mass.) and the exposed fracture planes were shadowed with Pt for 30 sec at an angle of 25-35 degrees followed with carbon for 35 sec (2 kV, 60-70 mA, 1×10⁻⁵ Torr). The replicas were cleaned with fuming HNO₃ for 24-36 hours followed by repeated agitation with chloroform/methanol (1:1 v/v) at least 5 times, and examined at a JEOL 100 CX electron microscope.

Radiolabeling of micelles. DTPA-PE-containing micelles in HBS were mixed with 50-100 μCi of ¹¹¹In in 0.1 M Na-citrate, pH 3.7. The mixture was incubated for 1 h at RT and dialyzed against at least 3000-fold excess of HBS overnight at 4° C. to remove unbound ¹¹¹In.

Binding of immunomicelles to substrate monolayers. ELISA plates were coated with 50 μl of 10 μg/ml nucleosomes (Worthington, Lakewood, N.J.) for testing 2C5-immunomicelles or with 50 μl of 10 μg/ml cardiac myosin for testing 2G4-immunomicelles and incubated overnight at 4° C. The rinsed plates were coated with 1% FBS in HBS. To substrate-coated plates, 50 μl of 2C5- or 2G4-immunomicelles at 20 μg/ml of PEG-PE were added and incubated for 4 h at RT. The plates were washed with HBS and coated with horseradish peroxidase-antimouse IgG conjugate (ICN Biomedicals, Aurora, Ohio) following the manufacturer's recommendations. The conjugate was removed after 3 h at RT, and the plates were washed with HBS. Bound peroxidase was quantified by degradation of diaminobenzidine (Neogen, Lexington, Ky.) supplied as a ready-for-use solution. Color intensity was analyzed by a Multiscan 340 ELISA reader (Labsystems, UK).

Interaction of 2C5—immunomicelles with cancer cells in vitro. After initial passage in tissue culture flasks, LLC and BT20 cells were grown on cover slips placed in 6-well tissue culture plates. After the cells reach a confluence of 60 to 70%, the plates were washed with Hank's buffer, and treated with 1% BSA in EMEM medium (2 ml/well) and incubated for 1 h at 37° C., 5% CO₂. To these cells, Rh-PE-labeled 2C5-immunomicelles were added to a final concentration of PEG-PE of 0.15 mg/ml and incubated for 1 h at 37° C., 5% CO₂. After incubation, the cells were washed with Hank's buffer, and the cover slips mounted cell-side down on glass slides using fluorescence free glycerol-based mounting medium (Fluoromount-G; Southern Biotechnology Associates, Birmingham, Ala.).

EL4 cells were grown in suspension to the density of about 2×10⁴ cells/ml, centrifuged at 700×g for 10 min, and transferred to Hank's buffer. The cells were washed and resuspended in Hank's buffer at about 1×10⁵ cells/ml density. Rh-PE-labeled immunomicelles were added to the EL4 cell suspension to a final PEG-PE concentration of 0.15-mg/ml. The cells were incubated with immunomicelles for 1 h at 37° C., 5% CO₂, washed with Hank's buffer, concentrated to a cell density of 1×10⁶ cells/ml, transferred to glass slides, and mounted as described. Mounted cells were studied with a Nikon Eclipse E400 microscope (Nikon, Japan) under bright light, or under epifluorescence with a rhodamine filter. 2C5-immunomicelles in vivo. All experiments were performed in 6-8 week old female C57BL/6J mice (Charles River Laboratories, Cambridge, Mass.) following protocol #011022 approved by the Institutional Animal Care and Use Committee in accordance with Principles of Laboratory Animal Care (NIH publication #85-23, revised in 1985). The animals were allowed free access to food and water.

For blood clearance experiments, the mice were injected with 100 μl of 0.5 mM ¹¹¹In-labeled micelle formulations via the tail vein. At the required time points between 0.5-17 h post injection, mice were anesthesized with ether and sacrificed by cervical dislocation. Blood was collected and analyzed for the presence of the micelle-associated ¹¹¹In radioactivity using a γ-counter (GAMMA 5500, Beckman, Fullerton, Calif.).

For tumor accumulation experiments, Lewis lung carcinoma (LLC) tumors were initiated in mice by subcutaneous injection of 20,000 LLC cells in 50 μL of 10 mM HBS into the left rear flank. When tumor diameters reached 3-7 mm (8-12 days post inoculation), the mice were injected with 100 μl of 0.5 mM ¹¹¹In-labeled micellar formulations via the tail vein. At 30 min and 2 h post injection, mice were sacrificed, tumors and muscle samples were collected and analyzed for the presence of ¹¹¹In radioactivity. There were 5 animals per group for each time point.

To estimate the accumulation of free and micellar taxol in tumors, LLC-bearing mice were injected with the same quantity of taxol in Cremophor EL/ethanol/saline mixture (see above) or in plain PEG-PE micelles or 2C5-immunomicelles (ca. 100 μg of taxol per animal). At 30 min and 2 h post injection, mice were sacrificed; tumors were removed, washed with saline and homogenized in the presence of 2.5 times the tumor weight of saline using a model 125 tissue homogenizer (PowerGen, Suwanee, Ga.). Taxol was extracted and quantified by HPLC as in (Sharma et al., 1994). tret-Butyl methyl ether used to extract taxol from homogenates contained 30 μg/mL of N-octylbenzamidine used as an internal HPLC standard and synthesized as in (Crosasso et al., 2000). The HPLC was run on a reverse phase Lichrospher RP18-5 column (Novato, Calif.) at flow rate of 0.9 ml/min with the detection, of taxol and the internal standard by optical density at 227 nm.

Inhibition of tumor growth with different taxol preparations. Mice with LLC tumors were injected with different taxol formulations on day 10 post inoculation (ca. 100 μg of taxol per animal per injection). On day 5 after the first administration, the injections were repeated. Twenty days after the first injection, the mice were sacrificed; tumors extracted, rinsed twice with saline, wiped, and weighed. There were five mice in each experimental group.

All experimental results are shown as mean values±standard deviations.

### Example I

### Exemplary Polymer Hicelles Loaded with Porphyrin

Porphyrin dissolved in methanol was added to a solution of PEG-PE in chloroform to obtain various final ratios of components. Organic solvents were removed under vacuum. Micelles were formed by shaking the PEG-PE/porphyrin film obtained in the presence of an aqueous buffer. Excess of porphyrin not incorporated into the micelles was separated by filtration of the micelle suspension through 0.2 μm filter. Concentration of porphyrin in micellar phase was estimated following the fluorescence at the excitation wavelength of 653 nm and the emission wavelength of 674 nm (F_{635/674}) after 100-200-fold dilution of the samples in methanol.

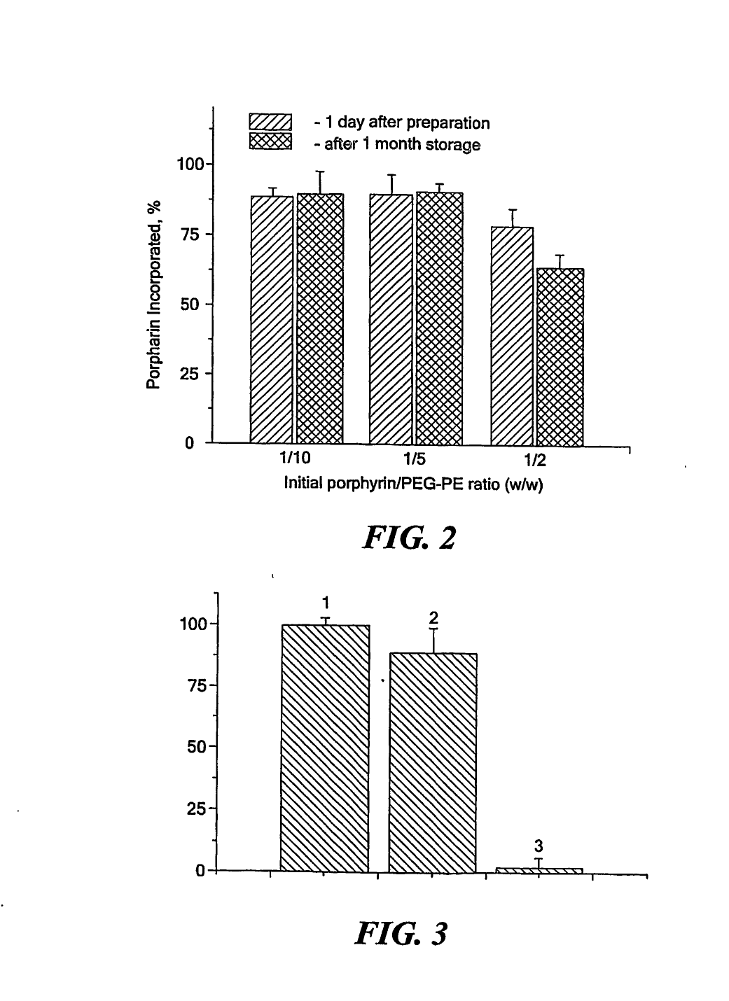

The results obtained are shown in FIG. 2. At initial porphyrin/PEG-PE weight ratio of up to 1/5, the agent incorporates into micelles with close to 100% efficiency. The efficiency decreases to about 80% at initial weight ratio of 1/2. In the latter preparation the drug/PEG-PE in the resultant micelles was as high as 2/5 w/w. The particles were stable for at least a month upon storage at room temperature. The data obtained demonstrate preparation of stable PEG-PE micelles with a high load of porphyrin.

### Example II

### Exemplary Polymer Hicelles Loaded with Tamoxifen

Tamoxifen dissolved in methanol was added to the solution of PEG-PE in chloroform to obtain the drug/PEG-PE molar ratio of 1:1. Organic solvents were evaporated and micelles were formed by shaking the tamoxifen/PE-PEG film obtained in the presence of an aqueous buffer at 50° C. Free tamoxifen was removed by filtration through 0.22 μm filters. Tamoxifen was quantified using the assay procedure for diethylstilbesterol (United States Pharmacopeal Convention, 2000).

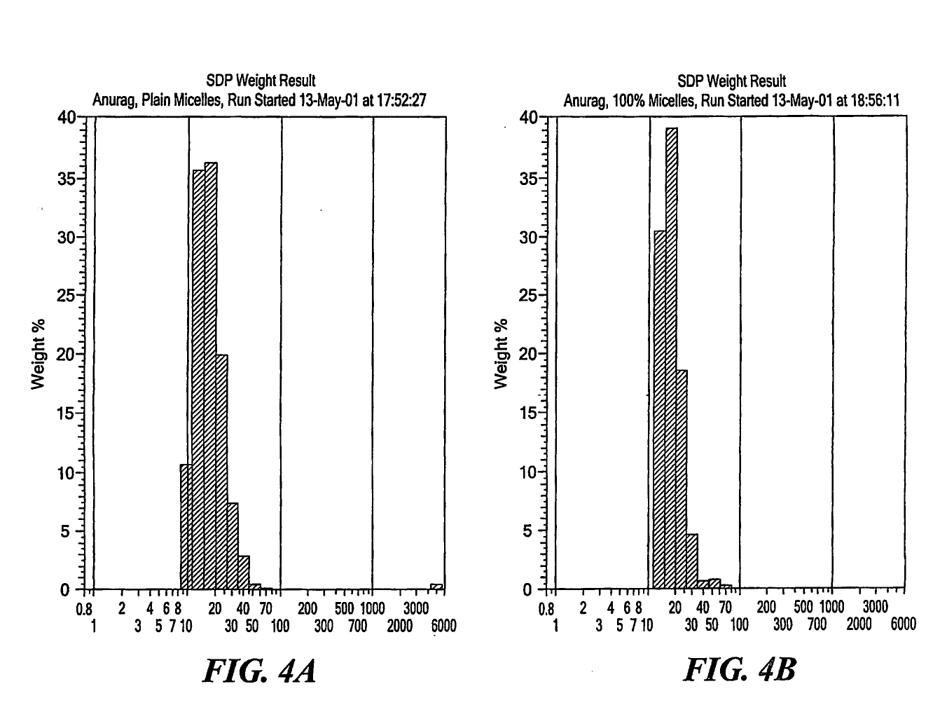

The results obtained are shown in FIG. 3. It can be seen that more than 95% of tamoxifen was incorporated into PEG-PE micelles. Incorporation of Tamoxifen does not change the size of the micelles significantly (FIG. 4). The results obtained demonstrate that PEG-PE micelles may be prepared with tamoxifen up to 1:1 drug/polymer molar ratio with preservation typical for the micelle size.

### Example III

### Preparation and Use of Targeted Micelles

Immunomicelles, a specific example of targeted micelles according to the invention, were prepared using a procedure (Torchilin et al., 2001; Torchilin, 2001). This procedure utilizes PEG-PE with the free PEG terminus activated with p-nitrophenylcarbonyl (pNP). Micelles were prepared from PEG-PE with the addition of a small fraction of pNP-PEG-PE. The PE residues form the micelle core, while pNP-groups allow for fast and efficient attachment of aminogroup-containing ligands via the formation of the urethane (carbamate) bond.

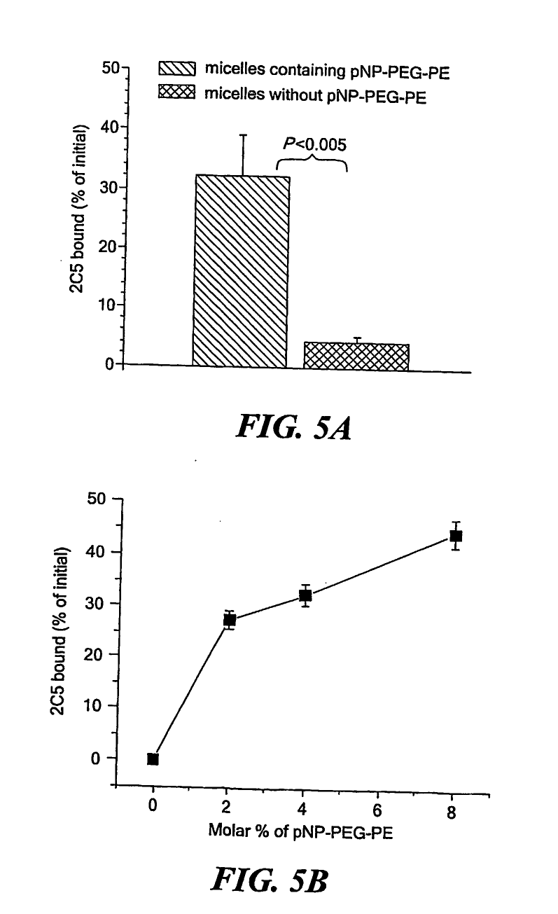

A typical result of micelle-bound antibody quantification is shown in FIG. 5. About 30% of added 2C5 attached to micelles containing 2 mol % of pNP-PEG-PE. This corresponds to about a 60% reaction yield because a 2-fold molar excess of protein over pNP-PEG-PE was used to avoid antibody inactivation due to modification of multiple NH₂-groups. This yield is close to the results obtained for liposomes in a similar procedure (Torchilin et al., 2001). From the yield value, it can be calculated that up to 10 antibody molecules bind to a single micelle. Protein binding to micelles without pNP-PEG-PE was negligible.

Antibody attachment yield may be increased by increasing the molar fraction of pNP-PEG-PE in micelles (FIG. 5). The yield reached as high as 50% when the micelles contained 8 mol % of pNP-PEG-PE. Excessive amount of pNP-PEG-PE, however, may cause over-modification of a protein molecule and its inactivation. Therefore, micelles with 2 mol % of pNP-PEG-PE were used to prepare immunomicelles for all further experiments.

One of the advantages of micelles as drug carriers is their small size. The transport and accumulation efficiency of microparticulates into the tumor interstitium is limited by their ability to penetrate tumor vascular endothelium (Jain, 1994; Yuan et al., 1995). Diffusion and accumulation of microparticles are determined by the cutoff size of the tumor vasculature, and this cutoff size varies for different tumors (Hobbs et al., 1998). If this size is below 200 nm, drug carriers with comparable or larger sizes (liposomes) will not extravasate (Parr et al., 1997). Thus, it is important that the modification of micelles with an antibody does not increase their size.

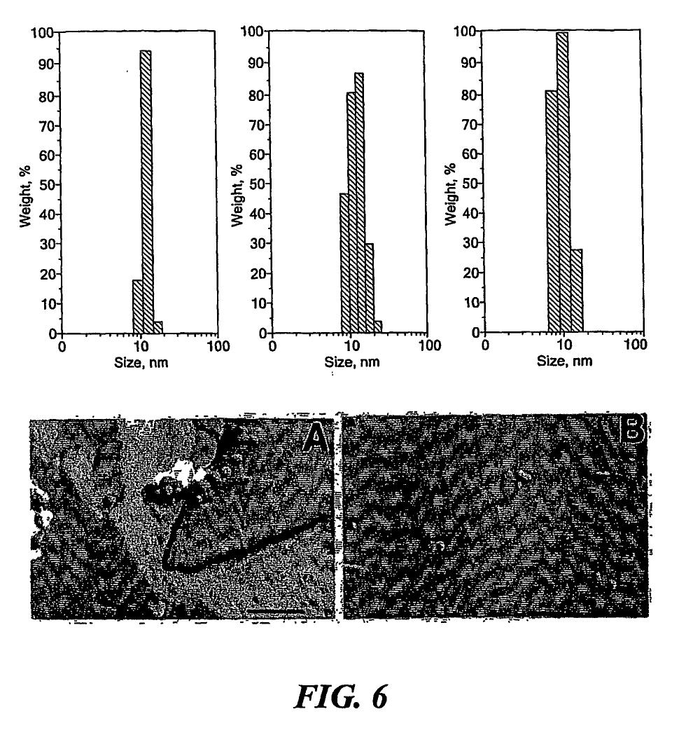

The size distribution of micelles before and after attachment of 2C5 or 2G4 is shown in FIG. 6. Protein attachment does not affect the micelle size significantly. Dynamic light scattering data were confirmed by the results of freeze-fracture electron microscopy (FIG. 6). Both the original and 2C5-modified micelles have a spherical shape, and a uniform size of about 20 nm. Thus, protein-modified micelles should retain the ability to cross the vasculature even of tumors with a relatively small cutoff sizes that are not accessible with other particulate delivery systems. (Gao et al., 2002; Weissig et al., 1998; Hobbs et al., 1998).

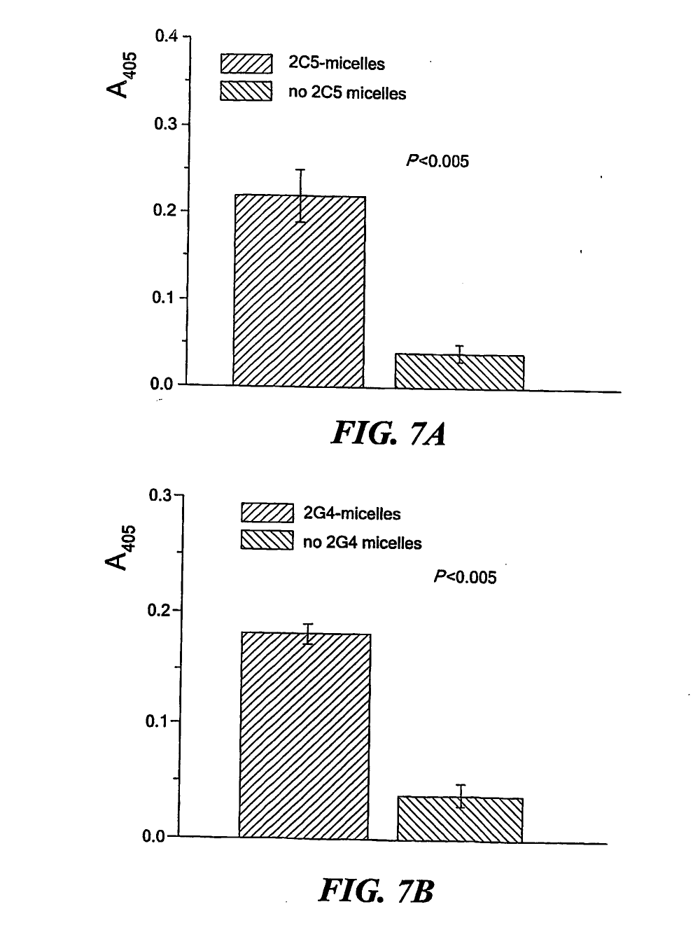

The binding of 2C5-immunomicelles to the 2C5 specific substrate, nucleosomes, and the binding of 2G4-immunomicelles to the 2G4 specific substrate, cardiac myosin, is shown in FIG. 7. Both 2C5- and 2G4-immunomicelles bound effectively to monolayers of corresponding antigens. The binding of control micelles (containing no PNP-PEG-PE but prepared following the same protocol and incubated with antibody) was much lower (only background binding can be seen because of some non-covalently adsorbed antibodies). The control with pNP-PEG-PE-containing antibody-free micelles is not required here, since pNP-groups spontaneously hydrolyze under experimental conditions yielding plain PEG-PE micelles. These results show that 2C5 and 2G4 antibodies preserved their specific activity upon attachment to the micelle surface.

To enhance tumor accumulation, drugs and drug carriers were modified with tumor-specific monoclonal antibodies (Goldenberg, 1993; Kemshead et al., 1993, Dillman, 2001). These antibodies are usually tumor type-specific and unable to react with different tumors. Recently, it was shown that certain nonpathogenic monoclonal antinuclear autoantibodies, with 2C5 among them, recognize the surface of numerous tumor, but not normal, cells via tumor cell surface-bound nucleosomes (Iakoubov et al., 1997; 1995; 1998). Because these antibodies bind a broad variety of cancer cells, they may serve as specific ligands for the delivery of drugs and drug carriers into tumors.

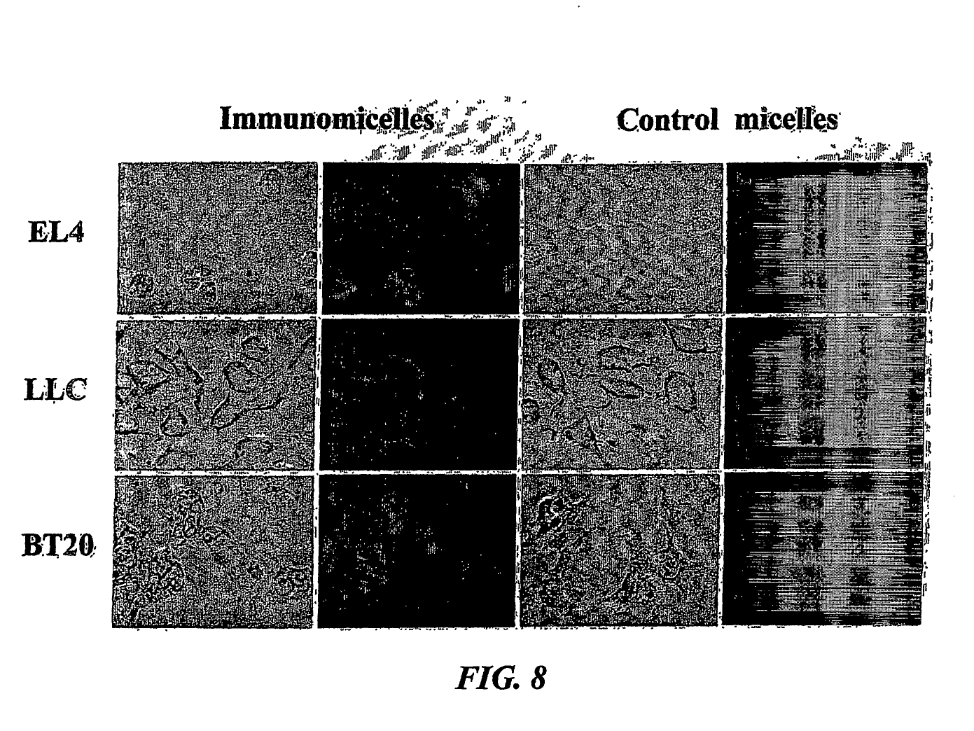

The results in FIG. 8 clearly show that rhodamine-labeled 2C5-immunomicelles effectively bind to the surface of several unrelated tumor cells lines: human BT20 and murine LLC and EL4 cells. The incubation of antibody-free micelles with the same cells results in virtually no micelle-to-cell association. No binding of 2C5 or 2C5-modified drug carriers with any normal cells was ever reported (Iakoubov et al., 1997). Thus, immunomicelles bearing an anti-nucleosomal antibody, which recognizes a variety of cancer cells, can specifically attach to these cells in vitro.

Prolonged circulation provides a drug carrier with a better chance to extravasate into the tumor interstitium and/or interact with ligands on the tumor cell surface. Direct correlation between the ability of a drug carrier to stay in the circulation and its accumulation in tumors was observed for water-soluble polymers (Maeda et al., 2001) and liposomes (Gabizon, 1995).

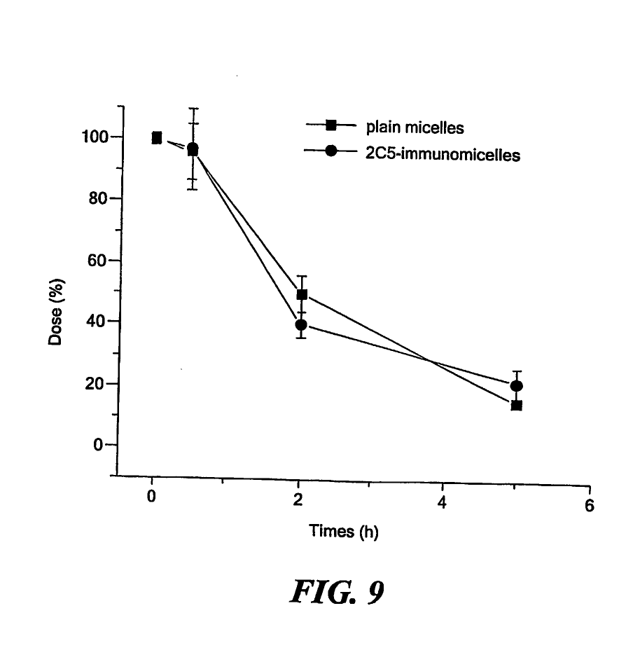

Micelles prepared from PEG-PE are long-circulating (Lukyonov et al., 2002). However, antibody attachment to drug carriers might provoke their faster clearance from the circulation due to uptake by Fc receptor-bearing Kupffer cells. To test whether the antibody attachment affects the blood clearance of PEG-PE micelles, clearance characteristics of plain and 2C5-modified micelles were compared in mice. The data shown in FIG. 9 clearly show that micelle modification with -2C5 had a very small effect on their blood clearance. Elimination profiles of plain and 2C5-modified micelles are almost identical. The data are consistent with earlier observations made with PEG-liposomes modified with anti-myosin antibodies (Torchilin et al., 1996).

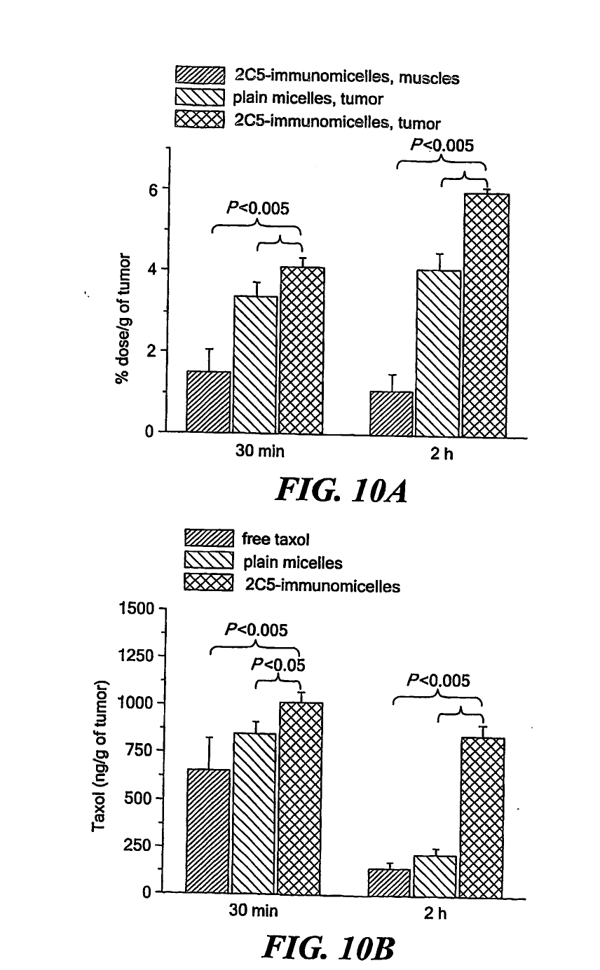

The EPR effect-mediated accumulation of drug carriers in tumors depends, among other factors, on tumor vasculature cutoff size (Jain, 1994). Low vascular permeability prevents many drug carriers including long-circulating liposomes from entering certain tumors, such as LLC (Hobbs et al., 1998; Parr et al., 1997). Since PEG-based micelles are much smaller than liposomes, they can provide an alternative and more efficient way of drug delivery (Weissig et al., 1998; Lukyanov et al., 2002). The accumulation of plain and immuno-PEG-PE-micelles was investigated in LLC tumor in mice. The results shown in FIG. 10 demonstrate that though PEG-PE micelles accumulate in the tumor much better than in normal tissue, their accumulation can be further improved if micelles are additionally modified with tumor-specific antibodies, i.e. converted into immunomicelles. 2C5-immunomicelles accumulate in LLC significantly better (by approx. 30%) than plain micelles at both investigated randomly chosen time points (FIG. 10). An enhanced accumulation of 2C5-targeted micelles over plain micelles in the tumor was observed at 30 min (4.1±0.2% dose/g of tumor vs 3.4±0.3% dose/g of tumor, P<0.05) and 2 h (6.4±0.1 dose/g of tumor vs 4.1±4 dose/g of tumor, P<0.005) post injection. Thus, 2C5-targeted micelles are capable of specific recognition and binding tumor cells in vivo. 2C5-immunomicelles are capable of delivering their load not only to tumors with a mature vasculature, but also to tumors at the earlier stages of their growth and to metastases. As shown, immunomicelles are better internalized by tumor cells similar to antibody-targeted liposomes (Park et al., 2001).

Experiments on taxol delivery into tumors have revealed an additional advantage of immunomicells. The use of taxol-loaded 2C5-micelles resulted in the highest quantity of tumor-accumulated taxol at both 30 min and 2 hr time points compared to free taxol or taxol in plain micelles. Interestingly, at 2 hr, bigger difference in accumulation of taxol in tumors with immunomicelles were seen compared to other taxol preparations than at 30 min. The difference is explained by the accumulation of free taxol or taxol delivered by plain PEG-PE micelles in the interstitial space of the tumor and its eventual clearance from there (in case of micellar taxol, after gradual micelle degradation). At the same time, taxol-loaded 2C5-immunomicelles were internalized by cancer cell and thus kept the drug inside the tumor in a way similar to what was observed with drug-loaded anti-her2 immunoliposomes (Park et al., 2001). The internalization by tumor cells would be highly useful therapeutically for many antitumor agents. For example, a much higher tumor regression rate was observed with a carrier capable of intracellular drug delivery for an equal doxorubicin dose delivered to the tumor (Park et al., 2001).

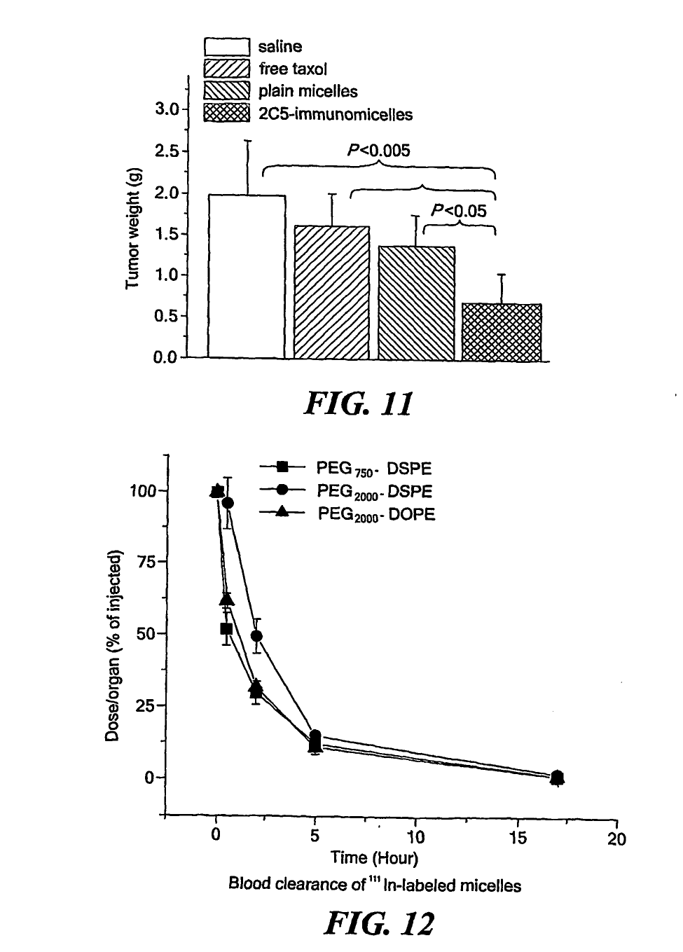

The results of the present proof-of-principle experiments on LLC growth inhibition by different taxol preparations have also confirmed the highest efficiency of taxol-loaded 2C5 immunomicelles among all tested preparations (see FIG. 11). Even within the model with a randomly chosen and non-optimized treatment schedule, the average weight of excised tumors in the group treated with taxol incorporated in immunomicelles was 0.67±0.35 g compared to 1.58±0.48 g and 1.37±0.36 g in groups treated with free taxol or taxol in plain PEG-PE micelles, respectively (P<0.05 in both cases). The weight of untreated tumors was 2.00±0.60 g. Taxol-free micelles of any composition did not affect tumor growth.

Thus, stable PEG-PE-based micelles with an enhanced ability to carry a variety of poorly soluble pharmaceuticals, can be transformed into immunomicelles by attaching various specific antibodies to their surface using the present method. These micelle-coupled antibodies preserve their specific activity. Immunomicelles prepared using cancer-specific 2C5 antibody specifically bind to different cancer cells in vitro and demonstrate increased accumulation in experimental tumors in vivo. This new family of pharmaceutical carriers should be very useful for the enhanced delivery of poorly soluble pharmaceuticals to various pathological sites in the body.

### Example IV

### Testing Different PEG-PE Micelles for an Efficient Tumor Accumulation in Various Tumor Models

The micelles were formed in HBS (5 mM HEPES, 150 mM NaCl, pH 7.4) by extensive 5-15 min vortexing of the lipid film prepared from distearoyl phosphatidylethanolamine poly-ethylene glycol 750 (PEG₇₅₀-PE) or distearoyl phosphatidylethanolamine poly-ethelenglycol 2000 (PEG₂₀₀₀-PE) at PEG-PE concentration of 5 mM. The micelles were labeled with ¹¹¹In via amphiphilic chelating agent, DTPA-PE, added to micelle composition.

Biodistribution and tumor accumulation of the ¹¹¹In-labelled micelles were studied in female C57B1/6J mice. Mice were inoculated subcutaneously with 20,000 LLC cells or 5,000 EL4 cells into left rear flanks (n=5 for each time-point). When tumor diameters reached ca. 0.5-1 cm (1-2 weeks post inoculation), the mice were injected with 100 μl of 0.5 M ¹¹¹In-labeled micellar formulations via the tail vein. At 0.5-17 h post injection, the mice were sacrificed by cervical dislocation; the organs of interest and implanted tumors were extracted and analyzed for the presence of micelle-associated ¹¹¹In radioactivity.

FIG. 12 shows the results of blood clearance experiments. All micelle formulations studied have circulation half-lives from 1.1 to 1.9 h depending on the molecular size of PEG block. This is a long half-life compared with the majority of non-surface-modified particulates (though somewhat shorter than that for PEG-coated liposomes). The shorter micelle half-life compared to PEG-liposomes may be explained by their faster extravasation from the vasculature due to their considerably smaller size when compared with liposomes (Weissig et al. 1998).

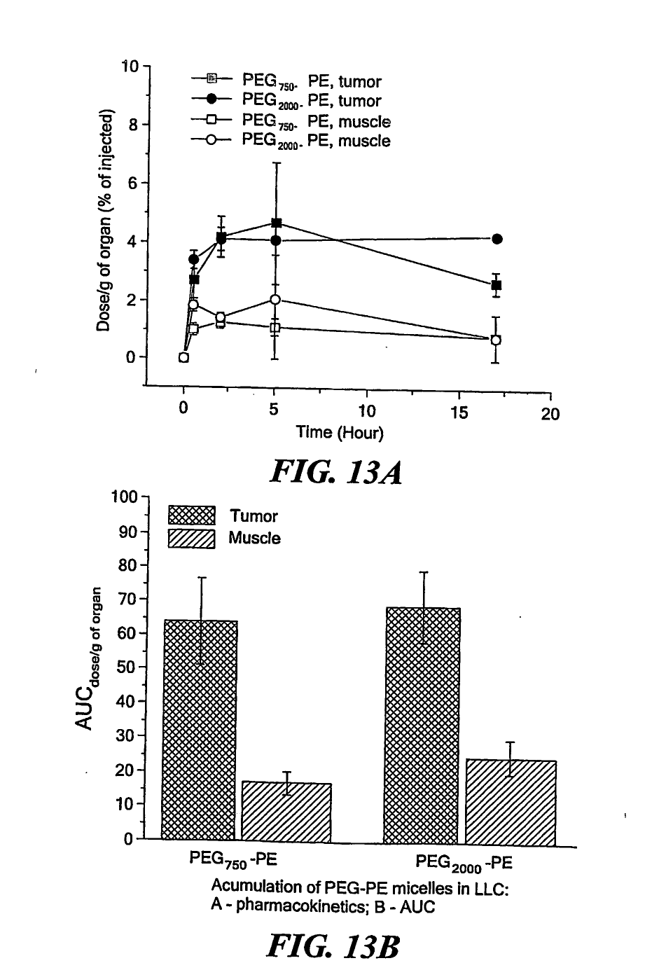

The data showing the accumulation of micelles prepared from PEG₇₅₀-PE and PEG₂₀₀₀-PE in LLC are presented in FIG. 13. Both formulations accumulate in the tumor more efficiently compared to the muscle tissue. Despite their slightly shorter half-life in the circulation, PEG₇₅₀-PE micelles have a higher targeting index (AUCₜᵤₘₒᵣ/AUC_{muscl}e) compare to PEG₂₀₀₀-PE; 3.8 and 2.8 respectively. PEG₂₀₀₀-PE micelles stay in the tumor for longer, however. No indication of their elimination was detected after as long as 17 h post-injection.

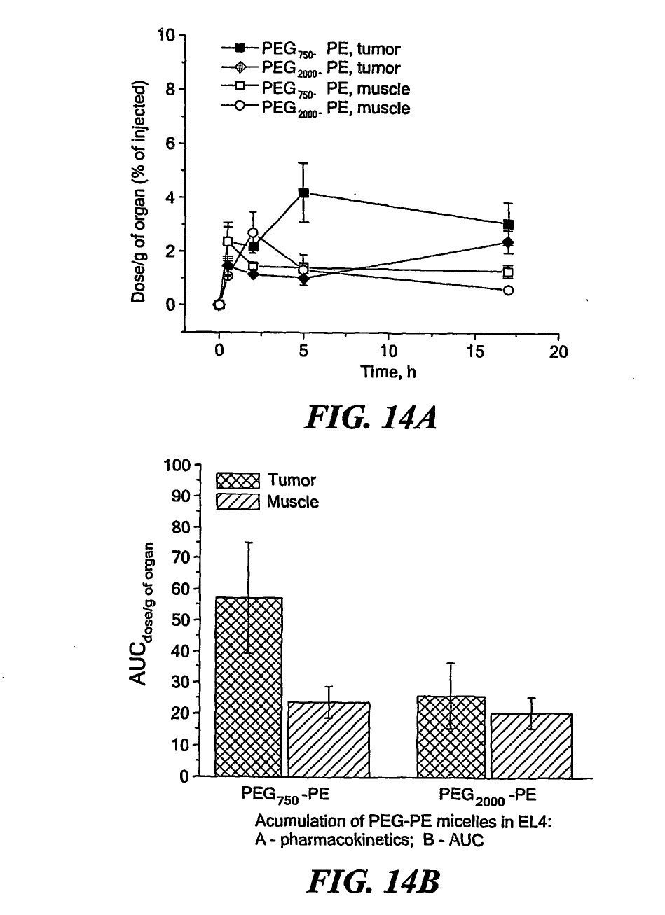

The accumulation of micelles in EL4 is shown in FIG. 14. In this case, only micelles made from PEG₇₅₀-PE exhibited preferential accumulation in the tumor (targeting index 2.4). One explanation is that the vasculature of this tumor has a smaller cut-off size and smaller PEG₇₅₀-PE micelles penetrate it more efficiently.

Characteristic size of the micelles of the invention makes them potentially a convenient delivery system for targeting pharmaceuticals utilizing enhanced permeability and retention (EPR) effect, which is based on a spontaneous penetration of the particles into the tumor interstitium through the leaky vasculature (Maeda et al. 2000). It has been shown that in some cases the use of particles with the diameter of 100 nm or larger does not allow utilizing of EPR effect in full, apparently because of small cutoff size of tumor vasculature (Yuan et al. 1995).

Therefore, micelles with very low critical micelle concentration (CMC) values prepared from lipidated polyethyleneglycols (PEG) of various lengths demonstrate long blood circulating half-life and accumulate selectively in various tumors, such as Lewis lung carcinoma (LLC) and EL4 T lymphoma (EL4), implanted in mice. PEG-PE micelles can effectively accumulate in tumors via EPR effect. The extent of their accumulation depends on the micelle size and cut-off size of the tumor vasculature.

### REFERENCES

Alkan-Onyuksel, H. et al., Material and methods for making improved micelle compositions. 2001: USA;
Allen, C., et al., Bioconjug. Chem., 9(5):564-72 (1998);
Allen et al. Coll. Surf. B:Biointerf., 16:1-35 (1999);
Arbuck, S. G., et al., J. Natl. Cancer Inst. Monogr., 15:11-24 (1993);
Burrows, F. J. et al., Cancer Res., 52(21):5954-62 (1992);
Cohen S. et al., New York: Marcel Dekker, Inc., (1996);
Crosasso, P. et al., J. Control. Release, 63:19-30 (2000);
Dalla Via et al., Curr. Med. Chem., 8(12):1405-18(2001);
Derossi, D., et al., J. Biol. Chem., 269(14):10444-50 (1994);
Dillman, R. O. Cancer Invest., 19:833-841 (2001);
Fawell, S., et al., Proc. Natl. Acad. Sci. U.S.A., 91(2):664-8 (1994);
Ferlini, C., et al., Br. J. Cancer, 75(6):884-91 (1997);
Fernandez, A. M. et al., J. Med. Chem., 44:3750-3753 (2001);
Furr, B. J. et al., Pharmacol. Ther., 25(2):127-205 (1984);
Gabizon, A. A., Adv. Drug Deliv. Rev., 16:285-294 (1995);
Gao, Z. et al., Nano Letters, 2:979-982 (2002);
Goldenberg, D. M., Am. J. Med., 94:297-312 (1993);
Grant, C. W. et al., Magn. Reson. Med., 11:236-243 (1989);
Hageluken, A. et al., Biochem. Pharmacol., 47:1789-1795 (1994)
Hammad, M. A. et al., Eur. J. Pharm. Sci., 7:49-55 (1998);
Hobbs, S. K. et al., Proc. Natl. Acad. Sci., USA 95:4607-4612 (1998);
Iakoubov, L. et al., Immunol. Lett., 47:147-149 (1995);
Iakoubov, L. Z. et al., Oncol. Res., 9:439-446 (1997);
Iakoubov, L. Z. et al., Cancer Detect. Prev., 22:470-475 (1998);
Jain, R. K., Sci. Am., 271:58-65 (1994);
Jeong, Y. I., et al., J. Control. Release, 51(2-3):169-78 (1998);
Johnston, S. R., Anticancer Drugs, 8(10):911-30 (1997);
Kabanov, A. V., et al., FEBS Lett., 258(2):343-5 (1989);
Kefalides, P. T., Ann. Intern. Med., 128(12 Pt 1):1053-5 (1998);
Kemshead, J. T. et al., J. R. Soc. Med., 86:219-224 (1993);
Khaw, B. A., et al., Hybridoma, 3(1):11-23 (1984);
Kim, S. Y., et al., J.. Control. Release, 51(1):13-22 (1998);
La, S. B. et al., J. Pharm. Sci., 85(1):85-90 (1996);
Lambert, D. M., Eur. J. Pharm. Sci., 11 Suppl 2:S15-27 (2000);
Langer, R., Nature, 392(6679 Suppl):5-10 (1998);
Lasic, D. D., Nature, 355:279-280 (1992);
Lasic, D. D. et al., Boca Raton: CRC Press (1995);
Layton, P. B. et al., J. Neurosurg, 60(6):1134-7 (1984);
Leroux, J.-C. et al., Labopharm. Inc: Canada (2000);
Lukyanov, A. N. et al., Pharm. Res., 19:1424-1429 (2002);
Maeda, H. et al., J. Control. Release, 65:271-84 (2000);
Maeda, H. et al., J. Control. Release, 74:47-61 (2001);
Mi, Z., et al., Mol. Ther., 2(4):339-47 (2000);
Miller, D. W., et al., Bioconjug. Chem., 8(5):649-57 (1997);
Muller, R. H., Boca Raton: CRC Press (1991);
Muranishi, S., Crit. Rev. Ther. Drug Carrier Syst., 7:1-33 (1990);
Palmer, T. N. et al., Biochim. Biophys. Acta, 797:363-368 (1984);
Park, J. W. et al., J. Control. Release, 74:95-113 (2001);
Parr, M. J. et al., J. Pharmacol. Exp. Ther., 280:1319-1327 (1997);
Phelan, A. et al., Nat. Biotechnol., 16(5):440-3 (1998);
Plank, C. et al., Adv Drug Deliv Rev., 34(1):21-35(1998);
Ramaswamy, M. et al., J. Pharm Sci., 86(4):460-4 (1997);
Rosen, M J. Surfactants and interfacial phenomena, 2^{nd} ed.; Wiley: New York, (1989);
Sarosy, G. et al., J. Natl. Med. Assoc., 85:427-431 (1993);
Senior, J. H., Crit. Rev. Ther. Drug Carrier Syst., 3:123-193 (1987);
Shabner B. A. et al., Philadelphia: J.B. Lippincott Co. (1990);
Sharma, A. et al., J. Chromatogr. B. Biomed. Appl., 655:315-319 (1994);
Songca, S. P. et al., J. Pharm. Pharmacol., 52(11):1361-7 (2000);
Torchilin, V. P. et al., Adv. Drug Deliv. Rev., 16:141-155 (1995);
Torchilin, V. P. et al., Biochim. Biophys. Acta, 1279:75-83 (1996);
Torchilin, V. P., Curr. Pharm. Biotechnol., 1(2):183-215 (2000);
Torchilin, V. P., Eur. J. Pharm. Sci., 11 Suppl 2:S81-91 (2000);
Torchilin, V. P. et al., Biochim. Biophys. Acta, 1511:397-411 (2001);
Torchilin, V. P. J. Control. Release, 73:137-172 (2001);
Torchilin, V. P., et al., Proc. Natl. Acad. Sci. U.S.A., 98(15):8786-91 (2001);
Trubetskoy, V. S. et al., Adv. Drug Deliv. Rev., 16:311-320 (1995);
Trubetskoy, V. S. et al., Acad. Radiol., 3:232-238 (1996);
The United States Pharmacopeal Convention, I., ed. The United States Pharmacopea. 24 ed. 2000: Rockville. 2569;
Vives, E. et al., J. Biol. Chem., 272(25):16010-7 (1997);
Wagner, E., Adv. Drug Deliv. Rev., 38(3):279-289 (1999);
Weissig, V. et al., Pharm. Res., 15:1552-1556 (1998);
Yokogawa, K. et al., Pharm. Res., 7:691-696 (1990);
Yokoyama, M., et al., Cancer Res., 50(6):1693-700 (1990);
Yokoyama, M., et al., .J Control Release, 50(1-3):79-92 (1998);
Yokoyama, M., et al., J Control Release, 55(2-3):219-29 (1998);
Yuan, F. et al., Cancer Res., 54:3352-3356 (1994); and
Yuan, F. et al., Cancer Res., 55:3752-3756 (1995).

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. A drug delivery system comprising:
a micelle comprising polyethylene glycol and a lipid component; and
a pharmaceutical agent dispersed in said lipid component.

2. The system of claim 1, wherein said pharmaceutical agent is selected from the group consisting of anti-inflammatory agents, imaging agents, agents for photodynamic therapy, anti-tumor agents, anti-neoplastic agents, anti-metastatic agents, and hydrophobized derivatives thereof.

3. The system of claim 1, wherein said pharmaceutical agent is selected from the group consisting of porphyrin, chlorine-6-trimethyl ester, tamoxifen, paclitaxel, 1,3-bis(2-chloroethyl)-1-nitrosourea, camptothecin, ellipticine, rhodamine, dequalinium, diphenylhexatriene, vitamin K3, diethylene triamine pentaacetic acid and functional derivatives thereof.

4. The system of claim 1, wherein said polyethylene glycol has a molecular weight between 500 and 10,000 daltons.

5. The system of claim 1, wherein said polyethylene glycol has a molecular weight between 1,000 to 8,000 daltons.

6. The system of claim 1, wherein said lipid is a diacyllipid.

7. The system of claim 6, wherein said diacyllipid is phosphatidylethanolamine.

8. The system of claim 1, wherein said micelle has a diameter in the range of 5 to 100 nm.

9. The system of claim 1, further comprising a targeting ligand attached to said micelle.

10. A drug delivery system comprising:
a micelle comprising polyethylene glycol and a lipid component;
a targeting ligand; and
a pharmaceutical agent dispersed in said lipid component.

11. The system of claim 10, wherein said pharmaceutical agent is selected from the group consisting of porphyrin, chlorine-6-trimethyl ester, tamoxifen, paclitaxel, 1,3-bis(2-chloroethyl)-1-nitrosourea, camptothecin, ellipticine, rhodamine, dequalinium, diphenylhexatriene, vitamin K3, diethylene triamine pentaacetic acid and functional derivatives thereof.

12. The system of claim 10, wherein said polyethylene glycol has a molecular weight between 1,000 and 8,000 daltons.

13. The system of claim 10, wherein said lipid is a diacyllipid.

14. The system of claim 13, wherein said diacyllipid is phosphatidylethanolamine.

15. The system of claim 10, wherein said targeting ligand is selected from the group consisting of a peptide; protein; enzyme; lectin; biotin; avidin; mono-, oligo, and polysaccharide; hormone; cytokine; and polyclonal and monoclonal antibody, including fragments thereof.

16. The system of claim 10, wherein said targeting ligand is selected from the group consisting of 2C5 antibody and 2G4 antibody.

17. A method of administering a targeted pharmaceutical agent to a patient, said method comprising the steps of:
a. providing the drug delivery system of claim 10; and
b. administering to a patient a therapeutically effective amount of said system, whereby said system is delivered to an affected tissue or organ of said patient, thereby enhancing the efficacy of said pharmaceutical agent.

18. The method of claim 17, wherein said pharmaceutical agent is selected from the group consisting of anti-inflammatory agents, imaging agents, agents for photodynamic therapy, anti-tumor agents, anti-neoplastic agents, anti-metastatic agents and hydrophobized derivatives thereof.

19. The method of claim 17, wherein said pharmaceutical agent is selected from the group consisting of porphyrin, chlorine-6-trimethyl ester, tamoxifen, paclitaxel, 1,3-bis(2-chloroethyl)-1-nitrosourea, camptothecin, ellipticine, rhodamine, dequalinium, diphenylhexatriene, vitamin K3, diethylene triamine pentaacetic acid and functional derivatives thereof.

20. A method of administering a pharmaceutical agent to a patient, said method comprising the steps of:
a. providing the drug delivery system of claim 1; and
b. administering to a patient, systemically or locally, a therapeutically effective amount of said system, thereby enhancing the efficacy of said pharmaceutical agent.

21. The method of claim 20, wherein said pharmaceutical agent is selected from the group consisting of anti-inflammatory agents, imaging agents, agents for photodynamic therapy, anti-tumor agents, anti-neoplastic agents, anti-metastatic agents and hydrophobized derivatives thereof.

22. The method of claim 20, wherein said pharmaceutical agent is selected from the group consisting of porphyrin, chlorine-6-trimethyl ester, tamoxifen, paclitaxel, 1,3-bis(2-chloroethyl)-1-nitrosourea, camptothecin, ellipticine, rhodamine, dequalinium, diphenylhexatriene, vitamin K3, diethylene triamine pentaacetic acid and functional derivatives thereof.
